(19) United States
(12) Patent Application Publication
(10) Pub. No.: US 2014/0127144 A1
(43) Pub. Date: May 8, 2014

(54) Flavour Modifying Compounds

(71) Applicant: Givaudan S. A., Vernier (CH)

(72) Inventors: Xiaogen YANG, West Chester, OH (US); Elisabetta LUBIAN, Zurich (CH); Harry RENES, Lelystad (NL); Alexander P. TONDEUR, Loosdrecht (NL); Stephan HAIBER, Naarden (NL); Xinping LIU, Valley Park, MO (US); Xun FU, Cincinnati, OH (US)

(73) Assignee: Givaudan S. A., Vernier (CH)

(21) Appl. No.: 14/151,144

(22) Filed: Jan. 9, 2014

Related U.S. Application Data
(63) Continuation-in-part of application No. PCT/EP2012/063908, filed on Jul. 16, 2012.
(60) Provisional application No. 61/508,389, filed on Jul. 15, 2011.

(51) Int. Cl. A23L 1/22; A61K 8/46; A61K 8/42

(52) U.S. Cl. CPC A23L 1/22091; A61K 8/42; A61K 8/46
USPC 424/54; 426/538; 426/534

(57) ABSTRACT

A compound of formula (I) is used to modify the taste or flavour of a flavour composition or consumable product,

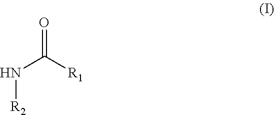

wherein R₁ is H, or a substituted, unsubstituted, branched or unbranched C₁-C₅ alkyl group and, NHR₂ is a residue of an amino acid, is selected from Alanine (Ala), cysteine (Cys), Aspartic acid (Asp), phenylalanine (Phe), glutamic acid (Glu), histidine (His), isoleucine (Ile), lysine (Lys), leucine (Leu), methionine (Met), asparagines (Asn), glutamine (Gln), arginine (Arg), serine (Ser), theronine (Thr), valine (Val), tryptophan (Trp), tyrosine(Tyr) and Glycine (Gly), with the proviso that the compound is not N-acetyl glycine. Also disclosed are flavour compositions and consumable products containing such compounds.

The present application is a continuation-in-part of international patent application PCT/EP2012/063908, filed Jul. 16, 2012, which claims priority from U.S. Provisional Patent Application Ser. No. 61/508,389, filed Jul. 15, 2011, and which applications are incorporated by reference herein.

In the flavour industry there is a constant demand for compounds that modify the taste or flavour of flavour compositions and consumable products. Such compounds extend a flavourist's palette and result in greater product diversity for consumers.

In particular, there is demand for compounds that modify the salt taste, umami taste, or savoury flavor of a flavour composition or consumable product. Such compounds may replace, or reduce reliance, on compounds conventionally used to modify salt taste, umami taste, or savoury flavour such as sodium chloride (NaCl) and monosodium glutamate (MSG), the use of which can be undesirable.

It has now been found that compounds of formula (I), as defined herein, may be used to modify the taste or flavour, in particular, the salt taste, umami taste, or savoury flavor, of a flavour composition or consumable product.

Disclosed is the use of compounds of formula (I), as defined herein, to modify the taste or flavour, in particular the salt taste, umami taste, or savoury flavour, of a flavour composition or consumable product.

Additionally disclosed are flavour compositions and consumable products comprising a compound of formula (I), as defined herein.

Further disclosed is a method of modifying the taste or flavour, in particular the salt taste, umami taste, or savoury flavour, of a flavour composition or consumable product comprising adding to said flavour composition or consumable product a compound of formula (I), as defined herein.

According to a first illustrative embodiment, there is provided the use of a compound of formula (I) to modify the taste or flavour of a flavour composition or consumable product,

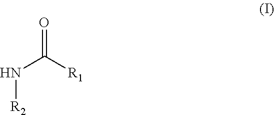

wherein

R₁ is H, or a substituted, unsubstituted, branched or unbranched C₁-C₅ alkyl group and, NHR₂ is a residue of an amino acid, selected from the group consisting of Alanine (Ala), cysteine (Cys), Aspartic acid (Asp), phenylalanine (Phe), glutamic acid (Glu), histidine (His), isoleucine (Ile), lysine (Lys), leucine (Leu), methionine (Met), asparagines (Asn), glutamine (Gln), arginine (Arg), serine (Ser), theronine (Thr), valine (Val), tryptophan (Trp), tyrosine (Tyr), Glycine (Gly), and mixtures thereof, with the proviso that the compound is not N-acetyl glycine.

According to a certain illustrative embodiments, when the amino acid, of which NHR₂ is a residue, comprises more than one amino group, the amino group that is bound to the carbonyl carbon shown in formula (I) is an amino group attached to the carbon atom alpha to the amino acid carboxylic acid group.

According to a certain illustrative embodiments, R₁ is H, or a substituted, unsubstituted, branched or unbranched C₁-C₅ alkyl group and, NHR₂ is a residue of an amino acid, selected from the group consisting of Glu, Phe, Ile, Lys, Leu, Val, Gly, and mixtures thereof, wherein when the amino acid, of which NHR₂ is a residue, comprises more than one amino group, the amino group that is bound to the carbonyl carbon shown in formula (I) is an amino group attached to the carbon atom alpha to the amino acid carboxylic acid group.

According to an illustrative embodiment, a compound of formula (I) is selected from the group consisting of N-acetyl-DL-glutamic acid, N-acetyl-L-glutamic acid, N-acetyl-methionine, N-formyl glycine, N-formyl-DL-phenylalanine, N-formyl-L-phenylalanine, N-acetyl-L-isoleucine, alpha-N-formyl-L-lysine, N-Hexanoyl-L-leucine, and N-pentanoyl-L-phenylalanine.

In an embodiment, the compound of formula (I) is N-acetyl-DL-glutamic acid.

In an embodiment, the compound of formula (I) is N-acetyl-L-glutamic acid.

In an embodiment, the compound of formula (I) is N-formyl glycine.

In an embodiment, the compound of formula (I) is N-acetyl-methionine.

Compounds of the formula (I) may be formed by known methods using commercially available starting materials, reagents and solvents.

In particular, compounds of formula (I) may be prepared by a process comprising the acylation of an amino acid, or by a process comprising the acylation of an amino ester followed by hydrolysis of the ester linkage.

Non limiting examples of common acylation agents include acids, acid chlorides and acid anhydrides.

Non limiting examples of acids that may be used as acylation agents include: formic acid, acetic acid, propionic acid, butyric acid, pentanoic acid, hexanoic acid.

Non limiting examples of acid chlorides that may be used as acylation agents include: acetyl chloride, propionyl chloride, butyroyl chloride, pentanoyl chloride, hexanoyl chloride.

Non limiting examples of acid anhydrides that may be used as acylation agents include: acetic anhydride, propionic anhydride butyric anhydride, pentanoic anhydride, hexanoic anhydride, or the mixed anhydrides.

Non limiting examples of hydrolysis agents include: diluted aqueous hydrochloric, sulphuric or phosphoric acid, diluted aqueous sodium hydroxide, sodium carbonate or potassium carbonate.

Compounds of formula (I) may exist as a mixture of stereoisomers or in isomerically pure forms. Resolution of sterioisomers can be carried out by techniques generally known in the art. The use of the term “a compound” of formula (I) may refer to both a racemic mixture and to pure sterioisomers.

The compounds of formula (I) may modify the taste or flavour of a flavour composition or consumable product by altering any element of the complex flavour.

The complex flavour is the combination of the olfactory, gustatory and trigeminal sensations sensed on consumption of a flavour composition or consumable product.

Non limiting examples of elements of the complex flavour include: salt taste, umami taste, sweet taste, kokumi taste, sour taste, bitter taste and savoury flavour.

The compounds of formula (I) can modify the taste or favour of a flavour composition or consumable product by imparting a taste or flavour, or by altering the perception of a taste or flavour.

The compounds of formula (I) can modify the taste or favour of a flavour composition or consumable product by modifying the salt taste, umami taste, or savoury flavour.

According to an embodiment there is provided the use of a compound of formula (I) to modify the salt taste, umami taste, or savoury flavor, of a flavour composition or consumable product comprising a salt tastant, umami tastant or savoury flavour compound.

The compounds of formula (I) may alter the perception of taste or flavour, in particular a salt taste, umami taste, or savoury flavour temporally, or by altering the quality or intensity for example by enhancing, strengthening, softening, sharpening, or making more salivating.

The temporal profile of a taste or flavour includes three aspects, the very first taste or flavour sensation (“initial impact”), the medium taste or flavour sensation (“body”), and the time period during which taste or flavour lasts or lingers (“lingering period”). Typically, the ‘initial impact’ lasts from 0 to 5 seconds, the ‘body’ lasts between 5 to 20 seconds, and the ‘lingering period’ lasts from 20 seconds onwards.

The compounds of formula (I) may modify any aspect of the temporal profile of the taste or flavour.

In particular, compounds of formula (I) can increase or draw out the initial impact or lingering period.

According to an embodiment, compounds of formula (I) may be used to increase or draw out the initial impact of the salt taste, umami taste or savoury flavour of a flavour composition or consumable product.

According to an embodiment, compounds of formula (I) may be used to increase or draw out the lingering period of the salt taste, umami taste or savoury flavour of a flavour composition or consumable product.

According to an embodiment, the initial impact or lingering period is drawn out or increased by 0.01 to 10 seconds, 1 to 5 seconds, 2 to 5 seconds.

The flavour composition or consumable products may comprise any known salt tastants, umami tastants, and savoury flavour compounds. Non limiting examples include: NaCl, KCl, MSG, guanosine monophosphate (GMP), inosin monophospahte (IMP), ribonucleotides such as disodium inosinate, disodium guanylate, N-(2-hydroxyethyl)-lactamide, N-lactoyl -GMP, N-lactoyl tyramine, gamma amino butyric acid, allyl cysteine, 1-(2-hydroxy-4-methoxylphenyl)-3-(pyridine-2-yl)propan-1-one, arginine, potassium chloride, ammonium chloride, succinic acid, N-(2-methoxy-4-methyl benzyl)-N′-(2-(pyridin-2-yl)ethyl) oxalamide, N-(heptan-4-yl)benzo(D)(1,3)dioxole-5-carboxamide, N-(2,4-dimethoxybenzyl)-N′-(2-(pyridin-2-yl)ethyl) oxalamide, N-(2-methoxy-4-methyl benzyl)-N′-2(2-(5-methyl pyridin-2-yl)ethyl) oxalamide, cyclopropyl-E,Z-2,6-nonadienamide.

In a particular embodiment, the flavour composition or consumable product comprises NaCl.

In another particular embodiment, the flavour composition or consumable product comprises MSG.

In a further particular embodiment, the flavour composition or consumable product comprises a ribonucleotide such as disodium inosinate, and disodium guanylate.

In a further particular embodiment, the flavour composition or consumable product comprises MSG and a ribonucleotide such as disodium inosinate, and disodium guanylate.

In a further particular embodiment, the flavour composition or consumable product comprises NaCl, MSG and a ribonucleotide such as disodium inosinate, and disodium guanylate.

The taste and flavour modifying properties, in particular the salt taste, umami taste or savoury flavour modifying properties, of the compounds of formula (I), enable the levels of the salt taste, umami taste and savoury flavour compounds, e.g. NaCl, MSG and ribonucleotides such disodium inosinate, and disodium guanylate, ordinarily added to flavour compositions and consumable products to be reduced if added in combination with a compound of formula (I).

According to an aspect, there is provided the use of a compound of formula (I) to reduce the quantity of NaCl, or MSG, or ribonucleotides, in a flavour composition or consumable product.

Compounds of formula (I) may be used to reduce up to 99.9% of the NaCl or MSG in a flavour composition or consumable product. However, more commonly compounds of formula (I) will be used to reduce 10-35%, 10-25%, of the NaCl, MSG, or ribonucleotides in a flavour composition or consumable product.

The compounds of formula (I) may modify the kokumi taste of a flavour composition or consumable product.

The term “Kokumi” is a term used in the flavour industry to describe characteristics such as salivating, continuity, mouthfulness, richness and thickness.

In another embodiment, there is provided the use of a compound of formula (I) to modify the kokumi taste of a flavour composition or consumable product.

The compounds of formula (I) may modify the kokumi taste of any type of flavour composition or consumable product. However, in particular the compounds of formula (I) may be used to modify the kokumi taste of sweet or savoury tasting flavour compositions or consumable products.

The compounds of formula (I) may modify the sweet taste of a flavour composition or consumable product.

The compounds of formula (I) may modify the sweet taste of any type of flavour composition or consumable product.

In another embodiment, there is provided the use of a compound of formula (I) to modify the sweet taste of a flavour composition or consumable product.

The compounds of formula (I) may modify the sour taste of a flavour composition or consumable product.

The compounds of formula (I) may modify the sour taste of any type of flavour composition or consumable product.

In another embodiment, there is provided the use of a compound of formula (I) to modify the sour taste of a flavour composition or consumable product.

In another aspect, there is provided a method of modifying the taste or flavour of a flavour composition comprising adding to said flavour composition, a compound of formula (I).

In an embodiment, there is provided a method of modifying the salt taste, umami taste, or savoury flavour of a flavour composition comprising a salt tastant, umami tastant or savoury flavour compound, comprising adding to said flavour composition, a compound of formula (I).

In an embodiment, there is provided a method of increasing or drawing out the initial impact of the salt taste, umami taste, or savoury flavour of a flavour composition comprising at least one salt tastant, umami tastant, or savoury flavour compound, comprising the step of adding to said flavour composition a compound of formula (I).

In an embodiment, there is provided a method of increasing or drawing out the linger period of the salt taste, umami taste, or savoury flavour of a flavour composition comprising at least one salt tastant, umami tastant, or savoury flavour compound, comprising the step of adding to said flavour composition a compound of formula (I).

In a particular embodiment, the flavour composition comprises NaCl.

In another particular embodiment, the flavour composition comprises MSG.

In a further particular embodiment, the flavour composition comprises a ribonucleotide such as disodium inosinate, and disodium guanylate.

In a further particular embodiment, the flavour composition comprises MSG and a ribonucleotide such as disodium inosinate, and disodium guanylate.

In a further particular embodiment, the flavour composition comprises NaCl, MSG and a ribonucleotide such as disodium inosinate, and disodium guanylate.

In another embodiment, there is provided a method of modifying the kokumi taste of a flavour composition comprising adding to said flavour composition a compound of formula (I).

In a particular embodiment, the flavour composition comprises a sweet flavour composition.

In a particular embodiment, the flavour composition comprises a savoury flavour composition.

In another embodiment, there is provided a method of modifying the sweet taste of a flavour composition comprising adding to said flavour composition a compound of formula (I).

In another embodiment, there is provided a method of modifying the sour taste of a flavour composition comprising adding to said flavour composition a compound of formula (I).

The compounds of formula (I) may be added into a flavour composition in neat form, or in a solvent, or they may first be modified, for example, by entrapment with an entrapment material such as for example polymers, capsules, microcapsules, nanocapsules, liposomes, precursors, film formers, absorbents such as for example by using carbon or zeolites, cyclic oligosaccharides and mixtures thereof, or they may be chemically bound to substrates which are adapted to release the compounds of formula (I) upon application of an exogenous stimulus such as light, enzymes, or the like.

A compound of formula (I) may be used as the sole flavouring component in a flavour composition. Alternatively, a compound of formula (I) may be employed in conjunction with other compounds of formula (I) and/or other flavourant ingredients known in the art, in particularly salt tasting, umami tasting, savoury flavour, or taste or flavour enhancers thereof.

Compounds of formula (I) may be used in a flavour compositions at a concentration of up to 100% by weight of the flavour components of the flavour composition. However, more commonly compounds of formula (I) will be used with other flavour ingredients, in particular salt tasting, umami tasting, savoury flavour and taste or flavour enhancers thereof, at a concentration of 0.01% to 99.9% by weight of the flavour components of the flavour composition.

Other flavourant ingredients that may be used in flavour compositions with compounds of formula (I) include, but are not limited to, natural flavours, artificial flavours, spices, seasonings, and the like. Exemplary flavouring ingredients include synthetic flavour oils and flavouring aromatics and/or oils, oleoresins, essences, distillates, and extracts derived from plants, leaves, flowers, fruits, and so forth, and combinations comprising at least one of the foregoing.

Exemplary flavour oils include, for example, spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavouring agents include artificial, natural and synthetic fruit flavours such as vanilla, and citrus oils including lemon, orange, lime, grapefruit, yazu, sudachi, and fruit essences including apple, pear, peach, grape, blueberry, strawberry, raspberry, cherry, plum, prune, raisin, cola, guarana, neroli, pineapple, apricot, banana, melon, apricot, ume, cherry, raspberry, blackberry, tropical fruit, mango, mangosteen, pomegranate, papaya and so forth. Additional exemplary flavours imparted by a flavouring agent include a milk flavour, a butter flavour, a cheese flavour, a cream flavour, and a yogurt flavour; a vanilla flavour; tea or coffee flavours, such as a green tea flavour, an oolong tea flavour, a tea flavour, a cocoa flavour, a chocolate flavour, and a coffee flavour; mint flavours, such as a peppermint flavour, a spearmint flavour, and a Japanese mint flavour; spicy flavours, such as an asafetida flavour, an ajowan flavour, an anise flavour, an angelica flavour, a fennel flavour, an allspice flavour, a cinnamon flavour, a chamomile flavour, a mustard flavour, a cardamom flavour, a caraway flavour, a cumin flavour, a clove flavour, a pepper flavour, a coriander flavour, a sassafras flavour, a savoury flavour, a Zanthoxyli Fructus flavour, a perilla flavour, a juniper berry flavour, a ginger flavour, a star anise flavour, a horseradish flavour, a thyme flavour, a tarragon flavour, a dill flavour, a capsicum flavour, a nutmeg flavour, a basil flavour, a marjoram flavour, a rosemary flavour, a bayleaf flavour, and a wasabi (Japanese horseradish) flavour; a nut flavour such as an almond flavour, a hazelnut flavour, a macadamia nut flavour, a peanut flavour, a pecan flavour, a pistachio flavour, and a walnut flavour; alcoholic flavours, such as a wine flavour, a whisky flavour, a brandy flavour, a rum flavour, a gin flavour, and a liqueur flavour; floral flavours; and vegetable flavours, such as an onion flavour, a garlic flavour, a cabbage flavour, a carrot flavour, a celery flavour, mushroom flavour, and a tomato flavour.

According to some embodiments, the other flavourant ingredients include aldehydes and esters such as cinnamyl acetate, cinnamaldehyde, citral diethylacetal, dihydrocarvyl acetate, eugenyl 49 formate, p-methylamisol, and so forth can be used. Further examples of aldehyde flavourings include acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavours), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), and the like.

Further examples of other flavourant ingredients can be found in “Chemicals Used in Food Processing”, publication 1274, pages 63-258, by the National Academy of Sciences.

Compounds of formula (I) can additionally be used in flavour compositions in conjunction with one or more ingredients or excipients conventionally used in flavour compositions, for example, carrier materials and other auxiliary agents commonly used in the art. Suitable excipients for flavour compositions are well known in the art and include, for example, without limitation, solvents (including water, alcohol, ethanol, oils, fats, vegetable oil, and miglyol), binders, diluents, disintegranting agents, lubricants, flavouring agents, coloring agents, preservatives, antioxidants, emulsifiers, stabilisers, flavour-enhancers, anti-caking agents, and the like.

Examples of such carriers or diluents for flavour compositions may be found in for example, “Perfume and Flavour Materials of Natural Origin”, S. Arctander, Ed., Elizabeth, N.J., 1960; in “Perfume and Flavour Chemicals”, S. Arctander, Ed., Vol. I & II, Allured Publishing Corporation, Carol Stream, USA, 1994; in “Flavourings”, E. Ziegler and H. Ziegler (ed.), Wiley-VCH Weinheim, 1998, and “CTFA Cosmetic

Ingredient Handbook”, J. M. Nikitakis (ed.), 1st ed., The Cosmetic, Toiletry and Fragrance Association, Inc., Washington, 1988.

Other suitable and desirable ingredients of flavour compositions are described in standard texts, such as “Handbook of Industrial Chemical Additives”, ed. M. and I. Ash, 2^{nd} Ed., (Synapse 2000).

In another aspect, there is provided a flavour composition comprising a compound of formula (I).

In an embodiment, there is provided a flavour composition comprising a compound of formula (I) and a salt tastant, umami tastant or savoury flavour compound.

In an embodiment, there is provided a flavour composition comprising a compound of formula (I) and NaCl.

In an embodiment, there is provided a flavour composition comprising a compound of formula (I) and MSG.

In an embodiment, there is provided a flavour composition comprising a compound of formula (I) and a ribonucleotide such as disodium inosinate, and disodium guanylate.

In an embodiment, there is provided a flavour composition comprising a compound of formula (I), MSG, and a ribonucleotide such as disodium inosinate, and disodium guanylate.

In an embodiment, there is provided a flavour composition comprising a compound of formula (I), NaCl, MSG, and a ribonucleotide such as disodium inosinate, and disodium guanylate.

In a further aspect, there is provided a method of modifying the taste or flavour of a consumable product comprising adding to said consumable product a compound of formula (I).

In an embodiment, there is provided a method of modifying the salt taste, umami taste, or savoury flavour, of a consumable product comprising a salt taste, umami taste or savoury flavour compound, comprising adding to said consumable product a compound of formula (I).

In an embodiment, there is provided a method of altering temporally the salt taste, umami taste, or savoury flavour of a consumable product comprising at least one salt tastant, umami tastant, or savoury flavour compound, comprising adding to said consumable product a compound of formula (I).

In an embodiment, there is provided a method of increasing or drawing out the initial impact of the salt taste, umami taste, or savoury flavour of a consumable product comprising at least one salt tastant, umami tastant, or savoury flavour compound, comprising adding to said consumable product a compound of formula (I).

In an embodiment, there is provided a method of increasing or drawing out the linger period of the salt taste, umami taste, or savoury flavour of a consumable product comprising at least one salt tastant, umami tastant, or savoury flavour compound, comprising adding to said consumable product a compound of formula (I).

In an embodiment, the consumable product comprises NaCl.

In an embodiment, the consumable product comprises MSG.

In an embodiment, the consumable product comprises a ribonucleotide such as disodium inosinate, and disodium guanylate.

In an embodiment, the consumable product comprises MSG and a ribonucleotide such as disodium inosinate, and disodium guanylate.

In an embodiment, the consumable product comprises NaCl, MSG and a ribonucleotide such as disodium inosinate, and disodium guanylate.

In another embodiment, there is provided a method of modifying the kokumi taste of a consumable product comprising adding to said consumable product a compound of formula (I).

In a particular embodiment, the consumable product is a sweet flavoured consumable product.

In another particular embodiment, the consumable product is a savoury flavoured consumable product.

In another embodiment, there is provided a method of modifying the sweet taste of a consumable product comprising adding to said consumable product a compound of formula (I).

In another embodiment, there is provided a method of modifying the sour taste of a consumable product comprising adding to said consumable product a compound of formula (I).

Compounds of formula (I), or flavour compositions containing compounds of formula (I), can be added to consumable products by using conventional techniques to directly admix said compound or flavour composition into the consumable product.

The quantities in which compounds of formula (I) may be added to consumable products may vary within wide limits and depend, inter alia, on the nature of the consumable product, on the effect desired, the purpose of adding compounds of formula (I) to a consumable product, for example creating or enhancing a salt taste, umami taste or savoury flavour, and on the nature and quantity of any other components of the consumable product. It is well within the purview of the person skilled in the art to decide on suitable quantities of compounds of formula (I) to incorporate into a consumable product depending on the end use and effect required.

Typical, non limiting, concentrations of compounds of formula (I), in ppm by weight based on the weight of the consumable product, are: 10,000 ppm to 0.01 ppm, more particularly 1000 ppm to 0.1 ppm, still more particularly 500 ppm to 1 ppm.

In another aspect, there is provided a consumable product comprising a compound of formula (I).

In an embodiment there is provided a consumable product comprising a compound of formula (I) and a salt tastant, umami tastant, or savoury flavour compound.

In an embodiment, there is provided a consumable product comprising a compound of formula (I) and NaCl.

In an embodiment, there is provided a consumable product comprising a compound of formula (I) and MSG.

In an embodiment, there is provided a consumable product comprising a compound of formula (I) and a ribonucleotide such as disodium inosinate, and disodium guanylate.

In an embodiment, there is provided a consumable product comprising a compound of formula (I), MSG, and a ribonucleotide such as disodium inosinate, and disodium guanylate.

In an embodiment, there is provided a consumable product comprising a compound of formula (I), NaCl, MSG, and a ribonucleotide such as disodium inosinate, and disodium guanylate.

The term “consumable product” as used herein refers to composition that may be placed in the oral cavity and ingested or that may be placed in the oral cavity before being discarded, for example mouthwash and chewing gum.

The compounds of formula (I) can be added to all manner of consumable products. Examples include, but are not limited to, foodstuffs of all kinds, confectionery products, baked products, sweet products, savoury products, fermented products, dairy products, beverages and oral care products.

Exemplary foodstuffs include, but are not limited to, chilled snacks, sweet and savoury snacks, fruit snacks, chips/crisps, extruded snacks, tortilla/corn chips, popcorn, pretzels, nuts, other sweet and savoury snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, uht soup, frozen soup, pasta, canned pasta, dried pasta, chilled/fresh pasta, noodles, plain noodles, instant noodles, cups/bowl instant noodles, pouch instant noodles, chilled noodles, snack noodles, dried food, dessert mixes, sauces, dressings and condiments, herbs and spices, spreads, jams and preserves, honey, chocolate spreads, nut-based spreads, and yeast-based spreads.

Exemplary confectionery products include, but are not limited to, chewing gum (which includes sugarized gum, sugar-free gum, functional gum and bubble gum), centerfill confections, chocolate and other chocolate confectionery, medicated confectionery, lozenges, tablets, pastilles, mints, standard mints, power mints, chewy candies, hard candies, boiled candies, breath and other oral care films or strips, candy canes, lollipops, gummies, jellies, fudge, caramel, hard and soft panned goods, toffee, taffy, liquorice, gelatin candies, gum drops, jelly beans, nougats, fondants, combinations of one or more of the above, and edible flavour compositions incorporating one or more of the above.

Exemplary baked products include, but are not limited to, alfajores, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savoury biscuits and crackers, and bread substitutes.

Exemplary sweet products include, but are not limited to, breakfast cereals, ready-to-eat (“rte”) cereals, family breakfast cereals, flakes, muesli, other ready to eat cereals, children's breakfast cereals, and hot cereals.

Exemplary savoury products include, but are not limited to, salty snacks (potato chips, crisps, nuts, tortilla-tostada, pretzels, cheese snacks, corn snacks, potato-snacks, ready-to-eat popcorn, microwaveable popcorn, pork rinds, nuts, crackers, cracker snacks, breakfast cereals, meats, aspic, cured meats (ham, bacon), luncheon/breakfast meats (hotdogs, cold cuts, sausage), tomato products, margarine, peanut butter, soup (clear, canned, cream, instant, UHT), canned vegetables, and pasta sauces.

Exemplary dairy products include, but are not limited to, cheese, cheese sauces, cheese-based products, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavoured, functional and other condensed milk, flavoured milk drinks, dairy only flavoured milk drinks, flavoured milk drinks with fruit juice, soy milk, sour milk drinks, fermented dairy drinks, coffee whiteners, powder milk, flavoured powder milk drinks, cream, yoghurt, plain/natural yoghurt, flavoured yoghurt, fruited yoghurt, probiotic yoghurt, drinking yoghurt, regular drinking yoghurt, probiotic drinking yoghurt, chilled and shelf-stable desserts, dairy-based desserts, and soy-based desserts.

Exemplary beverages include, but are not limited to, flavoured water, soft drinks, fruit drinks, coffee-based drinks, tea-based drinks, juice-based drinks (includes fruit and vegetable), milk-based drinks, gel drinks, carbonated or non-carbonated drinks, powdered drinks, alcoholic or non-alcoholic drinks.

Exemplary fermented foods include, but are not limited to, cheese and cheese products, meat and meat products, soy and soy products, fish and fish products, grain and grain products, fruit and fruit products.

In a particular embodiment, the consumable product is selected from the group consisting of Soy sauce, cheese, soup, hot and cold sauces, fruits, vegetables, ketchups, tea, coffee, snacks such as potato chips or extruded snacks.

There now follows a series of non-limiting examples that serve to illustrate the invention. The following examples are intended to merely illustrate the flavour compositions, consumable products, compounds, and methods, and are not intended to limit the scope of the claims in any manner.

### EXAMPLE 1

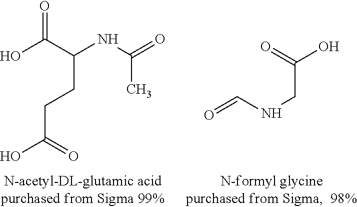

### EXAMPLE 1a

### Application on Soy Sauce

Mixture A: 5% of a standard soy sauce in water,

Mixture B: Same as mixture A plus 150-300 ppm N-acetyl-DL-glutamic acid plus 50-200 ppm N-formyl-glycine.

A panel of 5 people (expert panel) described mixture B as being more umami and more lingering savoury. This effect could be assigned to N-acetyl glutamic acid. Furthermore, mixture B showed a stronger initial salt spike, was more long-lasting, complex and more succulent compared to mixture A. This effect could be assigned to N-formyl glycine.

Using only 50-200 ppm N-formyl-glycine on top of mixture A (no N-acetyl-DL-glutamic acid) the mixture was described as more salty, more complex, long lasting and succulent compared to pure mixture A.

Using only 50 -300 ppm of N-acetyl-L-glutamic acid on top of mixture A (no N-formyl glycine) the effect was described as more umami and more lingering savoury.

### EXAMPLE 1b

### Application on Bechamel Sauce with Cheese

Mixture A: A Bechamel sauce with cheese was prepared by heating up 25 g of wheat flour in 35 g of butter in a saucepan. Let the flour cook shortly. Add 500 g milk, 2.5 g table salt, 0.15 g MSG, 0.075 g nutmeg powder, 0.125 g black pepper and 40 g of cheese powder. Boil the mixture for one minute.

Mixture B: The same as mixture A plus 150-300 ppm N-acetyl-DL-glutamic acid plus 50-200 ppm N-formyl-glycine.

A panel of 5 people (expert panel) stated that mixture B shows a stronger initial bite, is more salty and more complex compared to mixture A. This effect could be assigned to N-formyl glycine. Furthermore, mixture B was described as being more lingering, and giving fullness, depth and aftertaste of dairy cheese products compared to mixture A. This effect could be assigned to N-acetyl glutamic acid.

Using only 50-200 ppm N-formyl glycine on top of mixture A (no N-acetyl-DLglutamic acid) resulted in more bite, more complex note and saltiness compared to pure mixture A.

Using only 150-300 ppm N-acetyl-DL-glutamic acid on top of mixture A (no N-formyl glycine) resulted in more fullness, depth and aftertaste of cheese.

### EXAMPLE 2

### N-formyl-DL-phenylalanine

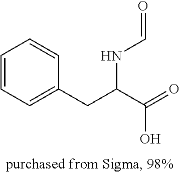 [SMILES: O=CNC(Cc1ccccc1)C(=O)O]

Mixture A: An aqueous solution of 0.5% NaCl

Mixture B: The same as A plus 10 ppm N-formyl-DL-phenylalanine

A panel of 5 people (expert panel) stated that mixture B is more salty.

Mixture C: An aqueous solution of 0.5% NaCl and 0.03% MSG.

Mixture D: The same as C plus 10 ppm N-formyl-DL-phenylalanine.

A panel of 5 people (expert panel) stated that mixture B is more umami.

### EXAMPLE 3

### N-acetyl-L-isoleucine

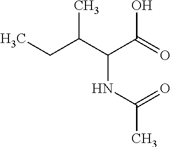

In a 250 ml round-bottomed flask was mixed isoleucine (15 g, 114 mmol) with acetic anhydride (31 g, 304 mmol) in methanol (50 ml) to give a white suspension. After 5.5 hours stirring at reflux, the reaction mixture was concentrated under reduced pressure.

Recrystallization from ethanol resulted in 13.3 g of white crystals.

The yield was 68.3% and the purity was higher than 98% according to NMR analysis.

1H-NMR in CDCl3/d6-DMSO: 0.505-0.648 (6H, d+t, CH3-CH2-CH(CH—NH—)—CH3), 0.823-0.958(1H, m, CH3-CH2-CH—), 1.069-1.235 (1H, m, CH3-CH2-CH—), 1.466-1.585 (1H, m, CH3-CH2-CH—), 1.624-1.719(3H, s, CH3-C═O), 4.053-4.156 (1H, m, R—HN—CH(COOH)CH), 6.792-6.903 (1H, d, R—NH)

13C-NMR in CDCl3/d6-DMSO:11.21 (1CH3, CH3-CH2-), 15.10 (1CH3, CH3-CH—), 22.50 (1CH3, CH3-CO—), 24.64 (1CH2, CH3-CH2-), 36.96 (1CH, CH3-CH—), 56.12 (1CH, R—CH—NH), 169.82 (1 Cq, CH3-CO—), 173.27 (—COOH)

Mixture A: An aqueous solution of 0.5% NaCl

Mixture B: The same as A plus 50 ppm of N-acetyl-L-isoleucine

A panel of 5 people (expert panel) stated that mixture B is more salty.

Mixture C: An aquesous solution of 0.5% NaCL and 0.03% MSG

Mixture D: The same as C plus 50 ppm of N-acetyl-L-isoleucine

A panel of 5 people (expert panel) stated that mixture D is more umami than mixture C.

### EXAMPLE 4

### alpha-N-formyl-L-lysine

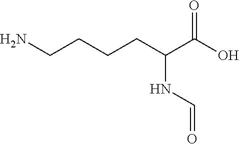 [SMILES: NCCCCC(NC=O)C(=O)O]

In a 250 mL round-bottomed flask was mixed L-lysine (6.6 g, 45.1 mmol) with formic acid (102.5 g, 2227 mmol) to give a white suspension. The resulting suspension was treated with acetic anhydride (24 g, 235 mmol) and stirred at room temperature for 1 hour. Then the solvents were removed on rotary evaporator. The residual thick oil was re-crystallized form ethanol and methyl tert-butyl ether to afford 1.7 g of white crystals. The yield was 17.7% and the purity was ca. 95% according to NMR analysis.

1H-NMR in D2O: 1.330-1.488, (2H, quin., H2N—CH2-CH2-), 1.650-1.720 (2H, m, H2N—CH2-CH2-CH2-), 1.720-1.786(1H, m, HOOC—CH(NH—CHO)CH2-), 1.786-1.935 (1H, m, HOOC—CH(NH—CHO)CH2-), 2.905-3.088 (2H, t, H2N—CH2-), 4.190-4.314 (1H, t, HOOC—CH), 8.021-8.128 (1H, s, CHO—NH—)

13C-NMR in D2O: 24.85 (1CH2, H2N—CH2-CH2-CH2-), 29.24 (1 CH2, H2N—CH2-CH2-), 34.00 (CH2, H2N—CH2-CH2-CH2-CH2-), 42.15 (1CH2, H2N—CH2-), 56.39 (1CH, HOOC—CH(NH—CHO)—CH2-), 166.59 (1CH, CHO—NH—), 181.26 (1Cq, HOOC—)

Mixture A: An aqueous solution of 0.5% NaCl and 0.03% of MSG.

Mixture B: The same as A plus 25 ppm N-formyl-L-lysine

A panel of 5 people (expert panel) stated that mixture B is more umami compared to mixture A.

### EXAMPLE 5

### N-hexanoyl-L-leucine

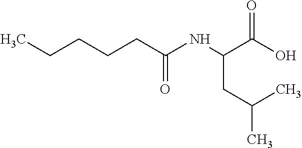 [SMILES: CCCCCC(=O)NC(CC(C)C)C(=O)O]

In a 500 mL round-bottomed flask, blanketed with nitrogen, hexanoyl chloride (6.88 g, 51.1 mmol) was added dropwise to a cold solution of (S)-ethyl 2-amino-4-methylpentanoate, HCl (10 g, 51.1 mmol) and triethylamine(15.67 ml, 112 mmol) in DCM (150 ml) with good stirring at temperature between 10 and 15° C. to give a colorless solution. The reaction mixture was allowed to stir at room temperature for 2 hrs then quenched with 1M HCl (200 ml). The organic was washed with 1M HCl (100 ml), sat NaCl (100 ml), and H2O (100 ml). The organic was dried with MgSO4, filtered and concentrated to yield 11.1 g of (S)-ethyl 2-hexanamido-4-methylpentanoate as colorless oil. Purity is >98% by NMR analysis.

In a 150 mL erlenmeyer flask was added a solution of (S)-ethyl 4-methyl-2-pentanamidopentanoate (10 g, 41.1 mmol) in ethanol (20 ml) to aqueous sodium hydroxide 5% solution (65.7 g, 82 mmol) and stirred at room temperature for 2 hrs. The reaction mixture was acidified to pH 1. The formed white precipitate was filtered, washed with water and pentane and then dried in vacuum oven at 50° C. 7.8 g of white powder was yielded. Purity is >98% by NMR analysis.

1H-NMR in CDCl3: 0.795-0.931 (3H, t, CH3-CH2-), 0.931-1.038 (6H, dd, CH3-CH(CH3)-CH2), 1.203-1.427 (4H, m, CH3-CH2-CH2-), 2.166-2.336 (5H, m, CH3CH(CH3)-, CH3-CH2-CH2-CH2, CH3CH(CH3)-CH2), 2.166-2.336 (2H, t, —CH2-CO—), 4.514-4.709 (1H, m, —CH(NH—)COOH), 5.929-6.123 (1H, b, —NH—)

13C-NMR in CDCl3: 13.88 (1CH3, CH3-CH2-), 21.86 (1CH2, CH3-CH2-), 22.32 (1CH3, CH3-CH(CH3)-), 22.80 (1CH3, CH3-CH(CH3)-), 24.88 (1CH2, CH3-CH2-CH2-CH2), 25.25 (1CH, CH3-CH(CH3), 31.30 (1CH2, CH3-CH2-CH2-), 36.42 (1CH2, -CH2-CO—), 41.14 (1CH, CH3-CH(CH3)-CH2-CH(NH—)COOH), 50.85 (1CH, —CH(NH—)COOH), 174.13 (1C, —NH—CO—), 176.46 (1C, CH(NH—)COOH

Mixture A: An aqueous solution of 0.5% NaCl, 0.03% MSG and 0.002%

Ribonucleotides GMP:IMP, ratio 1:1)

Mixture B: The same as A plus50 ppm of N-hexanoyl-L-leucine

An panel of 5 (expert panel) stated that mixture B is more salty and umami compared to mixture A.

### EXAMPLE 6

### N-pentanoyl-L-phenylalanine

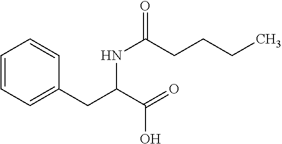 [SMILES: CCCCC(=O)NC(Cc1ccccc1)C(=O)O]

In a 500 mL round-bottomed flask blanketed with nitrogen, pentanoyl chloride (5.25 g, 43.5 mmol) was added dropwise to a solution of (S)-ethyl 2-amino-3-phenylpropanoate HCl (10 g, 43.5 mmol) and triethylamine (9.69 g, 96 mmol in DCM (150 ml with good stirring at room temperature to give a white suspension. The reaction mixture was allowed to stir at room temperature for 2 hrs then quenched with 1M HCl (200 ml). The organic was washed with 1M HCl (100 ml and water (100 ml). The organic was dried MgSO4 filtered and concentrated to yield 9.2 g of (S)-ethyl 2-pentanamido-3-phenylpropanoate as colorless oil. Purity >95% by NMR.

In a 150 mL Erlenmeyer flask was added a solution of (S)-ethyl 2-pentanamido-3-phenylpropanoate (8 g, 28.8 mmol) in methanol (10 ml) to aqueous sodium hydroxide 5% solution (69.2 g, 87 mmol) and stirred at room temperature for 1.5 hr. The reaction mixture was washed with 200 ml of ether and then acidified to pH 2. The formed white solid was filtered, washed with water, pentane and then dried in vacuum oven at 50° C. 2.8 g of white solid was yielded. Purity is >98% by NMR analysis.

1H-NMR in d6-DMSO:: 0.666-0.903 (3H, t, CH3-CH2-), 0.977-1.215 (2H, sextet, CH3-CH2-), 1.215-1.492 (2H, quin, CH3-CH2-CH2-), 1.872-2.130 (2H, quin, CH3-CH2-CH2-CH2-), 2.719-2.909 (1H, dd, —CH2-CH(NH)COOH), 2.936-3.133 (1H, dd, —CH2-CH(NH)COOH), 4.231-4.556 (1H, m, —CH2-CH(NH)COOH), 6.880-7.396 (5H, m C6H5-CH2-), 7.877-8.195 (1H, d, —NH—)

13C-NMR in d6-DMSO: 13.68 (1CH3, CH3-CH2-), 21.56 (1CH2, CH3-CH2-), 27.32 (1CH2, CH3-CH2-CH2-), 36.83 (1CH2, CH3-CH2-CH2-CH2-CO—) 53.40 (1CH, (—CH2-CH(NH)COOH) 126.26 (1CH, aromatic ring, para to substituent), 128.06 (2CH, aromatic ring, ortho to substituent), 129.08 (2CH, aromatic ring, metha to substituent), 137.89 (1C, aromatic ring), 172.08 (1C, —NH—CO—CH2-), 173.32 (1C, —CH—COOH)

Mixture A: An aqueous solution of 0.5% NACl, 0.03% MSG and 0.002%

Ribonucleotides (1:1 GMP, IMP)

Mixture B: The same as A plus 50 ppm of N-pentanoyl-L-phenylalanine

An panel of 5 (expert panel) stated that mixture B is more salty and umami compared to mixture A.

### EXAMPLE 7

### N-acetyl-DL-valine

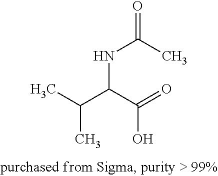

Mixture A: An aqueous solution of 0.5% NaCl

Mixture B: The same as A plus 10 ppm N-acetyl-DL-valine

A panel of 5 people (expert panel) stated that mixture B is more initially salty than mixture B.

Mixture C: An aqueous solution of 0.5% NaCl and 0.03% MSG (Mono sodium glutamate)

Mixture D: The same as C plus 75 ppm of N-acetlyl DL-valine

A panel of 5 people (expert panel) stated that mixture D is more lingering umami and salivating than mixture C.

### EXAMPLE 8

### N-acetyl-L-tyrosine

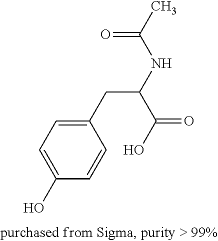 [SMILES: CC(=O)NC(Cc1ccc(O)cc1)C(=O)O]

Mixture A: A chicken broth

Mixture B: The same as A plus 100 ppm of N-acetyl-L-tyrosine

A panel of 5 people (expert panel) stated that mixture B is more salty, lingering umami and shows delayed numbing compared to mixture A.

### EXAMPLE 9

### N-acetyl-methionine (2-acetamido-4-(methylthio)butanoic acid)

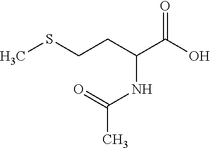

Methionine (5.7 g) was dissolved in 75 ml of 2 N NaOH and 50 ml of THF. Acetyl chloride was dissolved in 25 ml of THF and added slowly to the methionine solution at 0° C. The reaction mixture was stirred for 3 more hours at room temperature. Then the solution was acidified with concentrated hydrolchlorid acid solution to pH=2 and extracted 3 times with 100 ml of ethyl acetate. The combined organic layers were washed with water and brine and dried over magnesium sulfate. Removal of the ethly acetate yielded 3.8 g (52%) of the product with a purity of 85%.

1H NMR data in D2O: (in ppm) 6.2 (1H, NH), 4.5 (1H, HCN), 2.6 (2H, SCH2), 2.3 (2H, CH2), 2.2 (3H, SCH3), 1.9 (3H, COCH3).

Mixture A: salt reduced Kikkoman soy sauce containing 9% salt

Mixture B: same as Mixture A plus 0.04 ppm N-acetyl-methionine

An expert panel stated that Mixture B is more salty and has more upfront salty taste than Mixture A.

Mixture C: commercially available beef bouillon

Mixture D: same as Mixture C plus 40 ppb N-acetyl-methionine

The soups were tasted warm by a group of expert tasters. They stated that Mixture D is more salty, longer lasting and lifts the garlic component compared to Mixture C.

### EXAMPLE 10

### N-acetyl glutamic acid

150 ppm of N-acetyl glutamic acid was thoroughly mixed into Philadelphia brand cream cheese. This product was tasted and compared with the original cream cheese by a panel of experienced tasters consisting of 8 people (5 men, 3 women aged 35-54).

The panel agreed that the product with the N-Ac-Glu tasted saltier, creamier, more umami, more body, lingering and mouth-coating.

### EXAMPLE 11

### N-acetyl glutamic acid

100 ppm of N-acetyl glutamic acid was dissolved in a common salad dressing (Remia met magere yoghurt). This product was tasted and compared with the original salad dressing by a panel of experienced tasters consisting of 8 people (5 men, 3 women aged 35-54).

The panel agreed that the product with the N—Ac—Glu tasted more balanced, less sour, fuller, salivating and more lingering.

### EXAMPLE 12

### N-acetyl glutamic acid

200 ppm of N-acetyl glutamic acid was dissolved in a common cooking oil (Becel kitchen).

This product was tasted and compared with the original cooking oil by a panel of experienced tasters consisting of 8 people (5 men, 3 women aged 35-54).

The panel agreed that the product with the N—Ac—Glu tasted more fatty, more creamy, more mouthcoating and gave a better buttery aftertaste.

### EXAMPLE 13

### N-acetyl glutamic acid

Hamburgers were prepared from 80% lean beef and 20% fat beef meat containing 0.5% salt, 0.15% black pepper and 0.15% onion powder and 150 ppm of N-acetyl glutamic acid. The hamburgers were baked without additional fat and compared with the same batch of meat to which no N-acetyl glutamic acid was added. This product was tasted and compared with the original hamburgers by a panel of experienced tasters consisting of 8 people (5 men, 3 women aged 35-54).

The panel agreed that the product with the N—Ac—Glu tasted more meaty, more succulent, more juicy, more fatty and more expanding in the mouth.

### EXAMPLE 14

### N-acetyl glutamic acid

A standard instant mashed potato powder (Albert Heijn huismerk) was prepared and mixed with 150 ppm of N-acetyl glutamic acid. This product was tasted and compared with the original mashed potatoes by a panel of experienced tasters consisting of 8 people (5 men, 3 women aged 35-54).

The panel agreed that the product with the N—Ac—Glu tasted more salty, more creamy and more full.

### EXAMPLE 15

### N-acetyl glutamic acid

Potato chips were prepared in house. A cheese sour cream flavour was prepared by mixing salt (1.5 weight % on the chips), MSG (0.05), whey powder (2.5), cheese powder (1.5), cream powder (0.2), buttermilk powder (0.4) and onion powder (0.2) The powder was applied on plain potato chips. This product was tasted and compared with the chips without N—Ac—Glu by a panel of experienced tasters consisting of 8 people (5 men, 3 women aged 35-54).

The panel agreed that the product with the N—Ac—Glu tasted more salty, more creamy, gave more mouthwatering and had increased cheese intensity.

### EXAMPLE 16

### N-acetyl glutamic acid

To a standard tomato soup (Albert Heijn huismerk) was added 100 ppm of N-acetyl glutamic acid. This product was tasted and compared with the original tomato soup by a panel of experienced tasters consisting of 8 people (5 men, 3 women aged 35-54).

The panel agreed that the product with the N—Ac—Glu tasted less sour, had more body, was more salivating and had a stronger cooked taste.

### EXAMPLE 17

### N-acetyl glutamic acid

To a standard cola (Pepsi or Coke cola) was added 100 ppm of N-acetyl glutamic acid. This product was tasted and compared with the original colas by a panel of experienced tasters consisting of 8 people (5 men, 3 women aged 35-54).

The panel agreed that the product with the N—Ac—Glu tasted slightly less acidic, more syrupy and more smooth.

### EXAMPLE 18

### N-acetyl glutamic acid

To a standard soy milk (Alpro) was added 100 ppm of N-acetyl glutamic acid. This product was tasted and compared with the original soy milk by a panel of experienced tasters consisting of 8 people (5 men, 3 women aged 35-54).

The panel agreed that the product with the N—Ac—Glu tasted slightly more sweet and masked some of the soy off-notes.

### EXAMPLE 19

### N-acetyl glutamic acid

To a coffee beverage (Starbuck's frappucino) was added 150 ppm of N-acetyl glutamic acid. This product was tasted and compared with the original coffee beverage by a panel of experienced tasters consisting of 8 people (5 men, 3 women aged 35-54).

The panel agreed that the product with the N—Ac—Glu tasted slightly more bitter, had reduced metallic retort notes and had increased carmellic notes.

### EXAMPLE 20

### N-acetyl glutamic acid

To a standard vanilla yoghurt (Campina) was added 100 ppm of N-acetyl glutamic acid. This product was tasted and compared with the original vanilla yoghurt by a panel of experienced tasters consisting of 8 people (5 men, 3 women aged 35-54).

The panel agreed that the product with the N—Ac—Glu tasted less sour, more intense vanilla, more creamy, lingering and had more body.

Although the embodiments have been described in detail through the above description and the preceding examples, these examples are for the purpose of illustration only and it is understood that variations and modifications can be made by one skilled in the art without departing from the spirit and the scope of the disclosure. It should be understood that the embodiments described above are not only in the alternative, but can be combined.

1. A method of modifying the taste or flavour of a flavour composition or consumable product comprising adding to said flavour composition or consumable product a compound of formula (I) 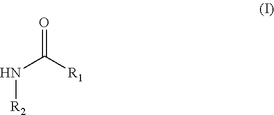
wherein
R₁ is H, or a substituted, unsubstituted, branched or unbranched C₁-C₅ alkyl group and, NHR₂ is a residue of an amino acid, selected from the group consisting of Alanine (Ala), cysteine (Cys), Aspartic acid (Asp), phenylalanine (Phe), glutamic acid (Glu), histidine (His), isoleucine (Ile), lysine (Lys), leucine (Leu), methionine (Met), asparagines (Asn), glutamine (Gln), arginine (Arg), serine (Ser), theronine (Thr), valine (Val), tryptophan (Trp), tyrosine(Tyr), Glycine (Gly), and mixtures thereof, with the proviso that the compound is not N-acetyl glycine.

2. The method according to claim 1, wherein the compound of formula (I) is selected from the group consisting of N-acetyl-DL-glutamic acid, N-acetyl-L-glutamic acid, N-acetyl-methionine, N-formyl glycine, N-formyl-DL-phenylalanine, N-formyl-L-phenylalanine, N-acetyl-L-isoleucine, alpha-N-formyl-L-lysine, N-Hexanoyl-L-leucine, N-pentanoyl-L-phenylalanine, and mixtures thereof.

3. The method according to claim 1, wherein the compound of formula (I) is selected from the group consisting of N-acetyl-DL-glutamic acid, N-acetyl-L-glutamic acid, N-acetyl-methionine, N-formyl glycine, and mixtures thereof.

4. The method according to claim 1, wherein said taste or flavour is selected from the group consisting of salt taste, umami taste, savoury flavour, sweet taste, sour taste and kokumi taste.

5. The method according to claim 4, wherein the salt taste, umami taste, or savoury flavour of the flavour composition or consumable product is altered temporally.

6. The method according to claim 5, wherein an initial impact or lingering period is increased or drawn out.

7. A flavour composition or consumable product comprising a compound of formula (I) 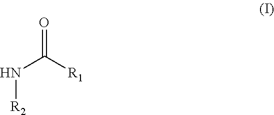
wherein
R₁ is H, or a substituted, unsubstituted, branched or unbranched C₁-C₅ alkyl group and, NHR₂ is a residue of an amino acid, selected from the group consisting of Alanine (Ala), cysteine (Cys), Aspartic acid (Asp), phenylalanine (Phe), glutamic acid (Glu), histidine (His), isoleucine (Ile), lysine (Lys), leucine (Leu), methionine (Met), asparagines (Asn), glutamine (Gln), arginine (Arg), serine (Ser), theronine (Thr), valine (Val), tryptophan (Trp), tyrosine(Tyr), Glycine (Gly), and mixtures thereof, with the proviso that the compound is not N-acetyl glycine;
and, at least one salt tastant, umami tastant, sweet taste, sour taste, or savoury flavour compound.

8. The flavour composition or consumable product according to claim 7, wherein the compound of formula (I) is selected from the group consisting of N-acetyl-DL-glutamic acid, N-acetyl-L-glutamic acid, N-acetyl-methionine, N-formyl glycine, N-formyl-DL-phenylalanine, N-formyl-L-phenylalanine, N-acetyl-L-isoleucine, alpha-N-formyl-L-lysine, N-Hexanoyl-L-leucine, N-pentanoyl-L-phenylalanine, and mixtures thereof.

9. The flavour composition or consumable product according to claim 7, wherein the compound of formula (I) is selected from the group consisting of N-acetyl-DL-glutamic acid, N-acetyl-L-glutamic acid, N-acetyl-methionine, N-formyl glycine, and mixtures thereof.

10. The flavour composition or consumable product according to claim 7, wherein said salt tastant, umami tastant or savoury flavour compound is selected from the group consisting of NaCl, MSG, and ribonucleotides.

11. The flavour composition or consumable product of claim 10, wherein said ribonucleotides are selected from disodium inosinate and disodium guanylate.

12. The consumable product according to claim 7, comprising a compound of formula (I) in a concentration of 0.01 to 10,000 ppm by weight based on the weight of the consumable product.

13. The consumable product according claim 7, selected from the group consisting of foodstuffs of all kinds, confectionery products, baked products, sweet products, savoury products, fermented products, dairy products, beverages and oral care products.

14. The consumable product according to claim 13, selected from the group consisting of soy sauce, cheese, soup, hot and cold sauces, fruits, vegetables, ketchups, tea, coffee, and snacks.

15. The consumable product of claim 14 wherein said snacks are selected from potato chips and extruded snacks.
